(19) United States
(12) Patent Application Publication
(10) Pub. No.: US 2026/0283603 A1
(43) Pub. Date: Sep. 24, 2026

(54) BIOPSY DEVICE AND METHOD OF PERFORMING A BIOPSY

(71) Applicant: BARD PERIPHERAL VASCULAR, INC., Franklin Lakes, NJ (US)

(72) Inventors: Bryon PELZEK, Gold Canyon, AZ (US); Andrzej J. CHANDUSZKO, Chandler, AZ (US)

(73) Assignee: BARD PERIPHERAL VASCULAR, INC., Franklin Lakes, NJ (US)

(21) Appl. No.: 19/479,198

(22) PCT Filed: Apr. 28, 2023
(86) PCT No.: PCT/US2023/020384

§ 371 Date: Oct. 27, 2025

(51) Int. Cl. A61B 10/02; A61B 17/32; A61B 17/34; A61B 18/00; A61B 18/12; A61B 18/14

(52) U.S. Cl. CPC A61B 10/0266; A61B 18/1477; A61B 2017/320064; A61B 17/3496; A61B 2018/00601; A61B 2018/0072; A61B 2018/126; A61B 2218/007

(57) ABSTRACT

A biopsy device includes an outer cannula having a body with a distal end, a proximal end, and at least one wall defining a passageway through the body, the distal end being open and having a sharpened edge. A first electrode and a second electrode are proximal the distal end of the body, and an inner needle is housed within the passageway defined by the outer cannula. The inner needle includes a piercing tip and is retractable from an extended configuration into the passageway to expose the sharpened edge of the outer cannula and allow the sharpened edge to cut a tissue core that is received by the passageway. An electrical pathway is formed between the first electrode and the second electrode when a voltage is applied between the first and second electrode to sever the tissue core.

### TECHNICAL FIELD

The present disclosure relates to biopsy devices, and, more particularly, to components for acquiring samples.

### BACKGROUND

A biopsy may be performed on a subject to help in determining whether tissue in a region of interest includes cancerous cells or other similarly diseased tissue. Traditional biopsy devices operate by utilizing a vacuum to pull tissue into a sample notch of the biopsy device, after which a cutting mechanism is utilized to sever the tissue from a target site. Although current biopsy devices may be effective in acquiring tissue samples from particular target sites, the tissue samples that may be acquired by these devices are limited by the size of the sample notch. Furthermore, traditional biopsy devices may struggle to acquire samples from a calcified target site, as the calcified tissue may cause the cutting mechanism to jam within the device.

Accordingly, a need exists for a biopsy device that is capable of acquiring relatively larger tissue samples and is more effective in acquiring samples from calcified or other similarly diseased target sites.

### SUMMARY

An object of the present disclosure is to provide a biopsy device that is capable of acquiring large tissue samples and is further able to easily acquire tissue samples from calcified and/or other similarly diseased target sites.

In one embodiment, a biopsy device is disclosed. The biopsy device includes an outer cannula having a body with a distal end, a proximal end, and at least one wall defining a passageway through the body, the distal end being open and having a sharpened edge. A first electrode and a second electrode are positioned proximal the distal end of the body, and an inner needle is housed within the passageway defined by the outer cannula. The inner needle includes a piercing tip that extends out of the distal end of the outer cannula in an extended configuration, the inner needle being retractable from the extended configuration into the passageway at a target site to expose the sharpened edge of the outer cannula to the target site such that the sharpened edge is configured to cut a tissue core, and the passageway is configured to receive the tissue core drawn into the passageway via the distal end. An electrical pathway is configured to be formed between the first electrode and the second electrode when a voltage is applied between the first and second electrode to sever the tissue core from the target site.

In another embodiment, a biopsy device is disclosed. The biopsy device includes an outer cannula having a body with a distal end, a proximal end, and at least one wall defining a passageway through the body, the distal end being open and having a sharpened edge. A first electrode and a second electrode are positioned proximal the distal end of the body, and an inner needle is housed within the passageway defined by the outer cannula. The inner needle includes a piercing tip that extends out of the distal end of the outer cannula in an extended configuration, and the inner needle is retractable from the extended configuration in the passageway at a target site to expose the sharpened edge of the outer cannula to the target site, such that the sharpened edge is configured to cut a tissue core and the passageway is configured to receive the tissue core drawn into the passageway via the distal end. A vacuum assembly having a sample basket is fluidly coupled to the proximal end of the outer cannula. An electrical pathway is formed between the first electrode and the second electrode when a voltage is applied between the first electrode and the second electrode to sever the tissue core at the target site.

In yet another embodiment, a method of performing a biopsy is disclosed. The method involves creating an incision with a retractable inner needle of a biopsy device, the biopsy device further comprising an outer cannula including a body having a proximal end, a sharpened distal end, and at least one wall defining a passageway disposed about the retractable inner needle. The method further involves retracting the retractable inner needle within the passageway of the outer cannula such that the sharpened distal end of the outer cannula is exposed. With the sharpened distal end exposed, the method may involve rotatably advancing the sharpened distal end of the outer cannula in a longitudinal direction into a target site such that the sharpened distal end cuts a tissue core and the passageway of the outer cannula receives the tissue core. Once the tissue core has been received by the outer cannula, the method may involve activating a first electrode and a second electrode embedded within an inner surface of the sharpened distal end of the outer cannula such that an electrical pathway is created between the first electrode and the second electrode, and severing the tissue core at the target site using the electrical pathway between the first electrode and the second electrode.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is a partial cross-sectional perspective view of a biopsy device, according to one or more embodiments described herein;

FIG. 2 is a partial cross-sectional top-side view of the biopsy device of FIG. 1, according to one or more embodiments described herein;

FIG. 3A is a cross-sectional front-side view of an inner needle of the biopsy device of FIG. 1 in an extended position, according to one or more embodiments described herein;

FIG. 3B is a cross-sectional front-side view of the inner needle of the biopsy device of FIG. 3A in a retracted position, according to one or more embodiments described herein;

FIG. 4A is a cross-sectional view of an outer cannula of the biopsy device of FIG. 1 in an initial position during a biopsy procedure, according to one or more embodiments shown and described herein;

FIG. 4B is a cross-sectional view of the outer cannula of the biopsy device of FIG. 4A in an acquisition position during a biopsy procedure, according to one or more embodiments shown and described herein;

FIG. 5 depicts a block diagram of illustrative internal components of a controller and a biopsy device according to one or more embodiments shown and described herein; and

FIG. 6 depicts an illustrative flow diagram of a method of performing a biopsy using the biopsy device of FIG. 1, according to one or more embodiments described herein.

Corresponding reference characters indicate corresponding parts throughout the several views. The exemplifications set out herein illustrate at least one embodiment of the present disclosure, and such exemplifications are not to be construed as limiting the scope of the present disclosure in any manner.

### DETAILED DESCRIPTION

Embodiments disclosed herein relate to biopsy devices and methods of performing biopsy procedures. For example, in embodiments, a biopsy device may include an outer cannula having a body with an open distal end having a sharpened edge, a proximal end, and at least one wall defining a passageway through the body. A first electrode and a second electrode may be diametrically opposed and embedded in an outer surface of the one wall. The biopsy device also includes an inner needle housed within the passageway that has a piercing tip that extends out of the open distal end of the outer cannula. The inner needle is retractable from the passageway to expose the sharpened edge of the outer cannula. In these embodiments, when the inner needle is retracted, the outer cannula may rotate and extend in a longitudinal direction, such that the sharpened edge extends into the target site and forms a tissue core. With the tissue core secured within the outer cannula, an electrical pathway may be formed between the first electrode and the second electrode to sever, or ablate, the tissue core from the target site. By rotatably extending the outer cannula into the target site, it may be possible to acquire larger tissue cores (e.g. in length, volume, etc.) via a single biopsy incision than those currently achieved by traditional biopsy devices. Furthermore, the rotation (e.g., manual or mechanical) of the sharpened distal edge of the outer cannula may allow for the biopsy device to be utilized in target sites that include calcified and/or particularly dense tissue that are prone to binding traditional biopsy devices.

Embodiments of biopsy devices and methods of performing biopsy procedures will now be described in more detail herein with reference to the drawings and where like numbers refer to like structures.

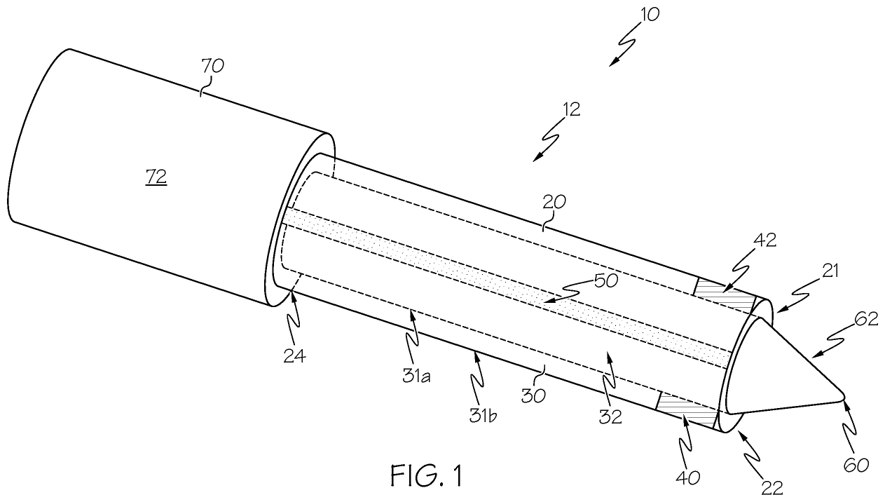

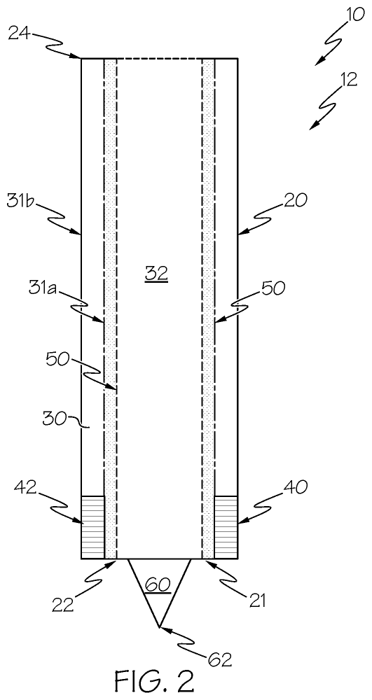

Referring now to FIGS. 1 and 2, a biopsy device 10 is depicted. The biopsy device 10 includes an outer cannula 12 including a body 20 having a distal end 22, a proximal end 24, and at least one wall 30 extending from the distal end 22 to the proximal end 24. In embodiments, the at least one wall 30 defines a passageway 32 extending through the body 20 from the distal end 22 to the proximal end 24. In the embodiments described herein, the distal end 22 of the body 20 includes a sharpened edge 21, such as a beveled edge, a serrated edge, or any other sharpened edge capable of coring tissue at a target site, as will be described in additional detail herein.

Referring still to FIGS. 1 and 2, the at least one wall 30 may include an inner surface 31a, which defines the passageway 32 through the body 20, and an outer surface 31b that is exposed to an external environment. In these embodiments, a plurality of electrodes, such as a first electrode 40 and a second electrode 42, may be integrated into the outer surface 31b of the at least one wall 30. For example, as depicted in FIGS. 1 and 2, the first electrode 40 and the second electrode 42 may be embedded within the outer surface 31b of the at least one wall 30. In other embodiments, the first electrode 40 and the second electrode 42 may be disposed on the outer surface 31b of the at least one wall 30, or otherwise integrated into the at least one wall 30. Furthermore, it should be appreciated that, although not depicted, the first electrode 40 and the second electrode 42 may be disposed on the inner surface 31a of the at least one wall 30 or on the distal end 22 of the body 20 without departing from the scope of the present disclosure. Although the biopsy device 10 depicted in FIG. 1 includes two electrodes, it should be understood that the plurality of electrodes may include any number of electrodes without departing from the scope of the present disclosure. For example, in some embodiments, the biopsy device 10 may include three electrodes, four electrodes, or any other number of electrodes.

In these embodiments, the first electrode 40 and the second electrode 42 may be embedded in the outer surface 31b of the at least one wall 30 such that the first electrode 40 and the second electrode 42 are diametrically opposed from one another. As defined herein, the term “diametrically opposed” may refer to an arrangement in which the first electrode 40 and the second electrode 42 are positioned on opposite sides of a target site, such as a tissue sample, such that the first electrode 40 and the second electrode 42 are aligned in a straight line through a center of the target site. In these embodiments, the first electrode 40 and the second electrode 42 may be disposed at opposite ends of the straight line, such that the first electrode 40 and the second electrode 42 are as far apart from one another as possible along the straight line that passes through the center of the target site. It should be appreciated that this alignment may allow for accurate and clean severing of a tissue sample from the target site, as will be described in additional detail herein.

Furthermore, the first electrode 40 and the second electrode 42 may be configured to be oppositely charged, such that the first and second electrodes 40, 42 are in a bipolar arrangement and an electrical pathway may be formed between the first electrode 40 and the second electrode 42 when tissue positioned at the target site contacts each of the respective electrodes. For example, in some embodiments, the first electrode 40 may be configured to be a positive (e.g., positively charged) electrode, while the second electrode 42 may be a configured to be a negative (e.g., negatively charged) electrode. Although the first electrode 40 is described herein as being a positive electrode and the second electrode is described as being a negative electrode, it should be understood that the first electrode 40 may be a negative electrode and the second electrode 42 may be a positive electrode without departing from the scope of the present disclosure.

Referring still to FIGS. 1 and 2, the biopsy device 10 may further include a pair of conductors 50, which may be embedded in the inner surface 31a of the at least one wall 30 of the body 20 or may run alongside the inner surface 31a of the at least one wall 30. The conductors may be connectable to voltage or current source. In these embodiments, a voltage may be applied by the voltage or current source to the first electrode 40 and second electrode 42, such that an electrical current, such as a cutting current, passes through the first electrode 40, the second electrode 42, and the pair of conductors 50. As will be described in additional detail herein, when a tissue sample positioned at the target site is positioned between the first electrode 40 and the second electrode 42, the tissue sample may act to complete an electrical circuit between the first electrode 40, the second electrode 42, and the pair of conductors 50, such that the cutting current passes through the tissue sample. As should be appreciated in view of the foregoing, the electrical pathway formed between the first and second electrodes 40, 42 may be a product of the cutting current that passes between the first and second electrodes 40, 42.

In these embodiments, the cutting current that passes between the first electrode 40 and the second electrode 42 may be any current that is sufficient to sever a tissue core from a target site. For example, the cutting current passing between the first electrode 40 and the second electrode 42 may act to sever the tissue core from the target site when the cutting current is greater than the threshold ablation current of the tissue core, wherein the threshold ablation current is the current above which ablation of the tissue occurs. As should be appreciated, when the cutting current is greater than the threshold ablation current of the tissue core, the tissue core may begin to burn, and ultimately, sever from the target site at the point where the first electrode 40 and the second electrode 42 contact the tissue core.

It should be further appreciated that, in the embodiments described herein, the cutting current may be directly proportional to the voltage supplied to the first and second electrodes 40, 42. Thus, it may be possible to increase the cutting current by increasing the voltage supplied to the first and second electrodes 40, 42. In these embodiments, the voltage supplied to the first and second electrodes 40, 42 may be adjusted until the cutting current is greater than the threshold ablation current of the tissue core, as has been described herein. For example, a cutting current between 100 mA-1000 mA, such as between 400 mA-600 mA, may be used to sever tissue commonly found at biopsy target site (e.g., breast tissue, etc.). However, it should be understood that the cutting current may be adjusted to account for a variety of tissue cores having varying electrical properties (e.g. due to density, moisture, calcification, etc.).

Referring still to FIGS. 1 and 2, it should be appreciated that the cutting current may be an alternating current (“AC”) or a direct current (“DC”) depending on the type of tissue core being obtained from the target site. For example, DC current (e.g., current having a continuous flow of electric charge in a single direction) may generate a constant cutting current, which may be beneficial for treating tissue cores that have a specific threshold ablation current that must be utilized to cleanly and accurately sever the tissue core. In contrast, AC current (e.g., current that periodically changes directions and causes a changing electric field) may be capable of generating a pulsating cutting current, which may be beneficial for targeting a smaller area of tissue while minimizing electrolysis in other regions of a target site.

In embodiments in which the cutting current is an AC current, a user may monitor a frequency of the generated cutting current in addition to the amplitude of the cutting current. For example, in some embodiments, a pulsating cutting current may be needed to safely sever tissue from a target site (e.g., sensitive tissue, tissue prone to electrolysis, etc.). In these embodiments, a cutting current having a frequency of between 300 kHz and 4 Mhz, such as between 400 kHz and 500 kHz, may be utilized to sever a tissue core from the target site, as frequencies within the ranges provided herein may minimize side effects (e.g., stimulation) that occur during a biopsy process. By ensuring that the frequency of the cutting current is greater than the impedance of the tissue at the target site, a user may ensure that the biopsy device 10 is capable of severing tissue at the target site without causing muscular stimulating effects within the target site.

Although the biopsy device 10 depicted in FIGS. 1 and 2 utilizes an electrical pathway formed between the first electrode 40 and the second electrode 42 to sever tissue from the target site, it should be understood that additional mechanisms may be used to sever tissue from the target site. For example, in some embodiments, the biopsy device 10 may include a plasma cutting mechanism, such as an argon plasma cutting mechanism, or a laser cutting mechanism without departing from the scope of the present disclosure.

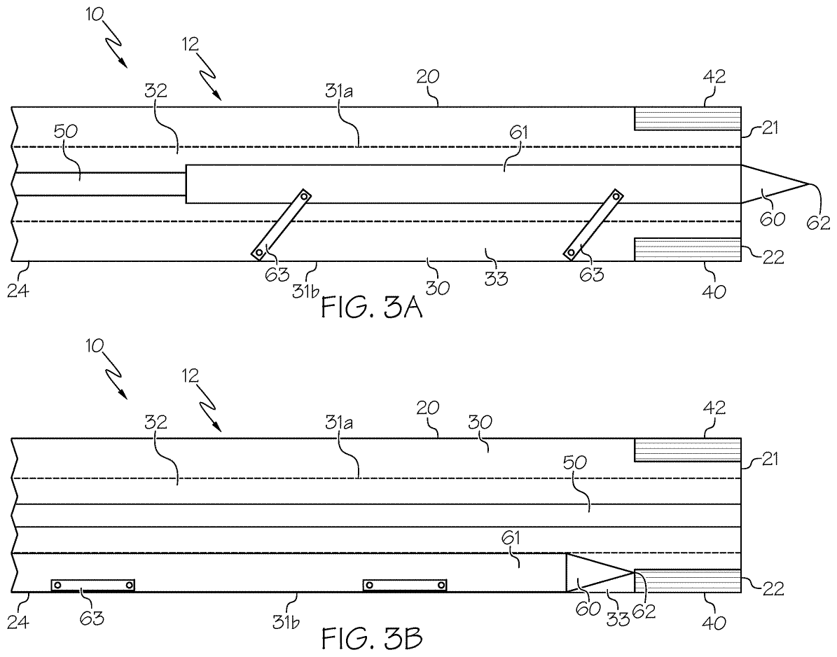

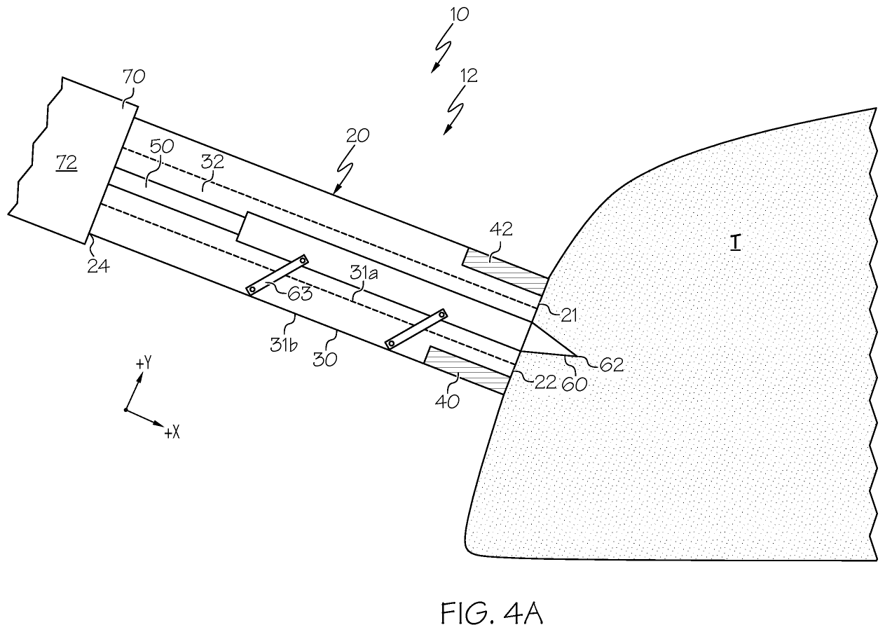

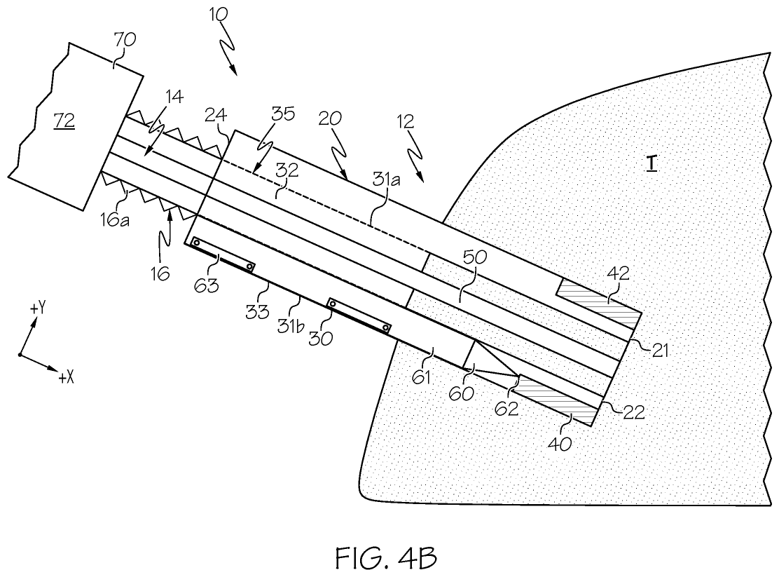

Referring still to FIGS. 1 and 2, the biopsy device 10 may further include a needle, such as an inner needle 60, having a piercing tip 62 that may be utilized to pierce tissue at the target site. As will be described in additional detail herein with reference to FIGS. 3A-4B, the inner needle 60 may be disposed on a plunger 61, or other similar device, that may be housed within the passageway 32 defined by the at least one wall 30 of the body 20 of the outer cannula 12. The plunger 61 may actuate the inner needle 60 between an extended position and a retracted position, such that the piercing tip 62 of the inner needle 60 extends beyond the distal end of the body 20 when the inner needle 60 is in the extended position and is retracted within the passageway 32 defined by the at least one wall 30 of the body 20 of the outer cannula when the inner needle 60 is in the retracted position.

Furthermore, the biopsy device 10 may include a vacuum assembly 70 that includes a sample basket 72. The vacuum assembly 70 is connectable to or comprises a vacuum or negative pressure source. In these embodiments, the sample basket 72 may be fluidly coupled to the proximal end 24 of the body 20 of the outer cannula 12, such that tissue cores acquired by the biopsy device 10 may be translated through the passageway 32 of the body 20 of the outer cannula 12 and into the sample basket 72 upon application of a vacuum or negative pressure at the vacuum assembly 70. For example, in these embodiments, the vacuum assembly 70 may be activated by generating a negative pressure at the vacuum assembly 70 to draw the tissue core through the passageway 32 and into the sample basket 72. Prior to activating the vacuum assembly, the inner needle 60 may be moved to the retracted position. In some embodiments, if necessary (for example if the size of the inner needle 60 is similar to the passageway 32, in the retracted position the inner needle 60 does not impede the passageway 32 of the body 20 as the negative pressure generated by the vacuum causes the tissue core to be translated through the passageway and disposed into the sample basket 72.

Turning now to FIGS. 3A and 3B, the inner needle 60 may be translated between the extended position and the retracted position via the plunger 61. In these embodiments, the at least one wall 30 of the body 20 of the outer cannula 12 may further include a recess, such as a slot 33, which may be configured to house the inner needle 60 when the inner needle 60 is in the retracted position. The plunger 61 may then force the inner needle 60 through the passageway 32 of the body 20 of the outer cannula 12 to move the inner needle to the extended position, such that the piercing tip 62 of the inner needle 60 extends beyond the distal end 22 of the body 20, as has been described herein.

As further depicted in FIGS. 3A and 3B, the plunger 61 may be coupled to the wall 30 via a plurality of arms 63, which may extend between the slot 33 and the plunger 61. In these embodiments, the plurality of arms 63 may be rotatably coupled (e.g., hingedly or otherwise) to the slot 33 at a first end and the plunger at a second end, such that rotation of the plurality of arms 63 may cause the plunger 61 (and in turn, the inner needle 60) to move between the extended position and the retracted position.

For example, FIG. 3A depicts the inner needle 60 in the extended position, in which the piercing tip 62 of the inner needle 60 extends beyond (e.g., in the +x-direction as depicted in the coordinate axis of FIG. 3A) the distal end 22 of the body 20 of the outer cannula 12. As further depicted in FIG. 3A, in the extended position, the plurality of arms 63 on which the plunger 61 is disposed may be rotated from the position shown in FIG. 3B in a first direction (e.g., clockwise) such that the plunger 61 is positioned outside of the slot 33, and extends through the passageway 32 of the body 20. As has been described herein, in the extended position, the piercing tip 62 may be utilized to pierce tissue positioned at the target site, such that the biopsy device 10 may access the target site and acquire a tissue core.

To ensure that the inner needle 60 remains in place when tissue positioned at the target site is pierced, the plurality of arms 63 coupled to the plunger 61 may include a locking mechanism, which may be used to lock the plurality of arms 63 in the extended position. For example, in some embodiments, the locking mechanism may include a catch, or latch, which engages at least one of the plurality of arms 63 when the plurality of arms 63 are in the extended position. In these embodiments, the latch may include a release mechanism position on the proximal end 24 of the outer cannula 12, which may be depressed by a user in order to release the plurality of arms 63 from the extended position. Similarly, the locking mechanism may include a pin, which may be insertable through the outer cannula 12 and at least one of the plurality of arms 63 when the plurality of arms are in the extended position. In these embodiments, the pin may secure the plurality of arms 63 in the extended position and may be removed to allow the plurality of arms 63 to be moved to the retracted position. By locking the plurality of arms 63, the plunger 61, and in turn, the inner needle 60, may be secured in the extended position, thereby allowing the inner needle 60 to pierce tissue positioned at the target site. Referring now to FIG. 3B, once the piercing tip 62 of the inner needle 60 has pierced tissue at the target site, the inner needle 60 may be translated to the retracted position, such that the piercing tip 62 of the inner needle 60 does not extend beyond the distal end 22 of the body 20 of the outer cannula 12. In these embodiments, to move the inner needle from the extended position to the retracted position, the plurality of arms 63 may be rotated in a second direction opposite the first direction (e.g., counterclockwise), such that the plunger 61 and the inner needle 60 are retracted into the slot 33. In these embodiments, when the inner needle 60 is moved to the retracted position, the piercing tip 62 may be positioned within the at least one wall 30, such that the inner needle 60 does not obstruct the passageway 32 of the outer cannula 12 during the biopsy process.

Although not depicted, it should be appreciated that the plurality of arms 63 may be rotated to translate the inner needle 60 using any manual, mechanical and/or electromechanical means. For example, a handle may be affixed to the inner needle 60 and connected to a rod or other elongate element extending along the outer surface 31b of the outer cannula 12, or through the wall 30 or along the inner surface of the wall 30, such that a user may grasp the handle (e.g. using a hand, a thumb and index finger, etc.) at a proximal end of the device and manually move the inner needle 60 between the extended position and the retracted position by movement of the handle. In other embodiments, an electromechanical mechanism, (e.g., actuator, spindle-gear set, etc.) may be utilized to translate the inner needle 60 between the retracted position and the extended position.

Referring still to FIG. 3B, when the inner needle 60 is moved to the retracted position, the sharpened edge 21 of the distal end 22 of the body 20 may be exposed to in a manner to engage with the target site. With the sharpened edge 21 of the distal end of the body 20 engageable, the outer cannula 12 may be advanced. As the outer cannula 12 is advanced, the sharpened edge 21 of the outer cannula 12 may sever tissue within the target site in order to create a tissue core within the passageway 32, as will be discussed in additional detail in reference to FIGS. 4A and 4B.

Although the inner needle 60 is depicted translated via the plunger 61 and plurality of arms 63, it should be appreciated that the inner needle 60 may be translated between the extended position and the retracted position through a variety of manual or electromechanical mechanisms. For example, the inner needle 60 may be coupled to the outer cannula via a magnetic coupling, such that as the outer cannula advances, a magnetic force may cause the inner needle to be retracted. In these embodiments, the inner needle 60 and outer cannula 12 may each include magnets that may be positioned such that the magnets attract each other when in close proximity. Accordingly, as the outer cannula 12 is advanced, the magnetic force between adjacent magnets may cause the inner needle 60 to be pulled back and/or retracted, such that the distal end 22 of the outer cannula 12 becomes exposed. Similarly, the inner needle may be retracted via a pneumatic and/or hydraulic actuator that may be activated by a user once the tissue at the target site has been pierced. It should be appreciated that the foregoing examples are provided for illustrative purposes only, and the inner needle 60 may be translated via any suitable mechanism. It should further be noted that retracting the inner needle 60 within the at least one wall of the outer cannula 12 after making an incision in a target site may allow for the biopsy device greater accessibility to tissue (e.g., lesions, etc.) positioned in areas that are otherwise difficult to access. For example, many areas within the chest of an individual (e.g., various areas of the breast, chest wall, skin surface, etc.) may be inaccessible to a biopsy device that includes an exposed needle, as the needle may cause damage to these areas. However, by retracting the inner needle 60 of the biopsy device 10 within the at least one wall 12 of the outer cannula 12, the biopsy device 10 may be more easily used to treat these areas.

Turning now to FIGS. 4A and 4B, the outer cannula 12 may be translatable between an initial position and an acquisition position. In these embodiments, the inner surface 31a of the outer cannula 12 may further include a screw thread 35, which may be coupled to an outer surface 16 of an inner cannula 14. In these embodiments the outer surface 16 of inner cannula 14 may include a threaded surface 16a, such that the screw thread 35 of the inner surface 31a of the outer cannula 12 is threadably coupled to the threaded surface 16a of outer surface 16 of the inner cannula 14. Accordingly rotating the body 20 of the outer cannula 12 may cause the outer cannula 12 to advance about the inner cannula 14 from the initial position and the acquisition position.

For example, rotating the body 20 of the outer cannula 12 in a first direction (e.g., a clockwise direction) may cause the outer cannula 12 to advance (e.g., in the +x-direction as depicted by the coordinate axes of FIGS. 4A-4B) from the initial position to the acquisition position, while rotating the body of the outer cannula 12 in a second direction opposite the first direction (e.g., a counterclockwise direction) may cause the outer cannula 12 to retract from the acquisition position to the initial position.

As has been described herein, in these embodiments, the inner surface 31a of the outer cannula 12 may be coupled to the outer surface 16 of the inner cannula 14 by way of threaded engagement. Accordingly, rotation of the outer cannula 12 relative to the inner cannula 14 may allow for the outer cannula 12 to retract and advance between the initial position and the acquisition position, as has been described herein.

In these embodiments, it should be further appreciated that the passageway 32 may extend through both the outer cannula 12 and the inner cannula 14, such that a tissue sample acquired by the biopsy device 10 may be transported through the outer cannula 12 and the inner cannula 14 and into sample basket 72. For example, in these embodiments, an outer diameter of the inner cannula 14 may be equivalent to an inner diameter of the outer cannula 12, such that the outer cannula 12 may be rotated about the inner cannula 14 (as has been described herein) while allowing the passageway 32 to extend through both the outer cannula 12 and the inner cannula 14 in an unobstructed manner.

As further depicted in FIGS. 4A and 4B, rotation of the outer cannula 12 about the inner cannula 14 may occur when the inner needle 60 has been moved to the retracted position. As has been described herein with reference to FIGS. 3A and 3B, the piercing tip 62 of the inner needle 60 may be utilized to create an incision in a target site. In these embodiments, the piercing tip 62 may create the incision in the target site prior to the outer cannula 12 being translated from the initial position to the acquisition position, as the sharpened edge 21 may be incapable of piercing the target site until the incision is created by the inner needle 60 of the piercing tip 62. For example, it may be necessary to create a relatively smaller incision using the piercing tip 62 of the inner needle 60 to allow the sharpened edge 21 of the at least one wall 30 to access the target site.

Although the outer cannula 12 is depicted as being translated via threaded engagement, it should be understood that the outer cannula 12 may be translated between the initial position and the acquisition position through a variety of manual or electromechanical mechanisms. For example, although not depicted, in some embodiments, the outer cannula 12 may be rotatably coupled to a proximal end of the device (for example vacuum assembly 70) via a rotary coupling, a bellows coupling, or any other suitable coupling that allows the outer cannula 12 to rotate. In these embodiments, the outer cannula 12 may be moved between the initial position and the acquisition position by grasping (e.g., using a hand, a thumb and index finger, etc.) the outer surface 31b of the outer cannula 12 and manually rotating the outer surface 31b of the outer cannula 12. In other embodiments, an electromechanical mechanism, (e.g., actuator, spindle-gear set, etc.) may be utilized to rotate the outer cannula 12 about the inner cannula 14, such that the outer cannula 12 translates between the initial position and the acquisition position.

Additionally, it should be noted that the outer cannula 12 may be rotated at a predetermined number of revolutions per minute (“RPM”) that may be based on the tissue located at the target site. In these embodiments, increasing the RPM at which the outer cannula 12 rotates may allow the biopsy device 10 to perform biopsy procedures at target sites that would be otherwise inaccessible. For example, target sites containing calcified or otherwise dense tissue may be impenetrable utilizing a traditional biopsy device. However, by increasing the RPM at which the outer cannula 12 rotates, the cutting efficacy of the biopsy device 10 may be improved, such that the biopsy device 10 is able to acquire tissue cores from areas of dense tissue.

Furthermore, as noted previously herein, the advancement of the outer cannula into the target site may allow for larger (e.g., in volume, length, etc.) tissue cores to be obtained from a target site. For example, traditional biopsy devices may be capable of obtaining tissue cores 2 cm in length. In contrast, the outer cannula 12 of the disclosed biopsy device 10 may be advanced about 4 cm into a target site, such that a larger tissue core may be obtained during a biopsy procedure. This may be particularly advantageous in conducting certain types of pathology analysis, which may require that a larger tissue core be obtained from a target site in order to properly determine the cause of disease and/or deterioration within the target site. With an ability to obtain a larger core, the device described herein may be utilized at a single site without the need for multiple rounds of tissue sampling, which can minimize surrounding tissue damage, multiple puncture sites, and/or the like.

A biopsy procedure will now be described with reference to FIGS. 4A and 4B. Initially, as shown in FIG. 4A, an incision may be made in a target site T using piercing tip 62 of the inner needle 60. When the piercing tip 62 is utilized to create the incision in the target site T, the inner needle 60 may be in the extended position and the outer cannula 12 may be in the initial position, such that the piercing tip 62 of the inner needle 60 extends beyond (e.g., in the +x-direction as depicted in the coordinate axes of FIGS. 4A-4B) the distal end 22 of the body 20 of the outer cannula 12.

Turning now to FIG. 4B, once the incision has been created in the target site T, the inner needle 60 may be retracted from the extended position to the retracted position. As the inner needle 60 is moved from the extended position to the retracted position, the sharpened edge 21 of the distal end 22 of the outer cannula 12 may be exposed in a manner to engage the target site T, such that the sharpened edge 21 contacts the target site T at a location where the incision is formed.

With the sharpened edge 21 of the distal end 22 of the outer cannula 12 in contact with the target site T and the inner needle 60 in the retracted position, the outer cannula 12 may be advanced (e.g., either manually or electromechanically) from the initial position to the acquisition position, in which the outer cannula 12 is used to acquire a tissue core.

As further depicted in FIG. 4B, rotation of the outer cannula 12 from the initial position to the acquisition position may cause the sharpened edge 21 of the distal end 22 of the outer cannula 12 to burrow into the target site T. As the outer cannula 12 continues to advance towards the acquisition position, a tissue core C may become enclosed within the passageway 32 of the outer cannula 12. In these embodiments, the outer cannula 12 may continue to advance from the initial position towards the acquisition position until a tissue core C of a desired volume is enclosed within the passageway 32 of the outer cannula 12.

When the tissue core C is enclosed within the passageway 32 of the outer cannula 12, a current may be applied between the electrodes and the electrical pathway formed between the first electrode 40 and the second electrode 42 through the tissue of the core C may act to sever the tissue core C from the target site T, such that the tissue core C may be removed from the target site. With the tissue core C severed from the target site T, the vacuum assembly 70 may be activated to draw the tissue core C into the sample basket 72, at which point the biopsy device 10 may be removed from the target site T.

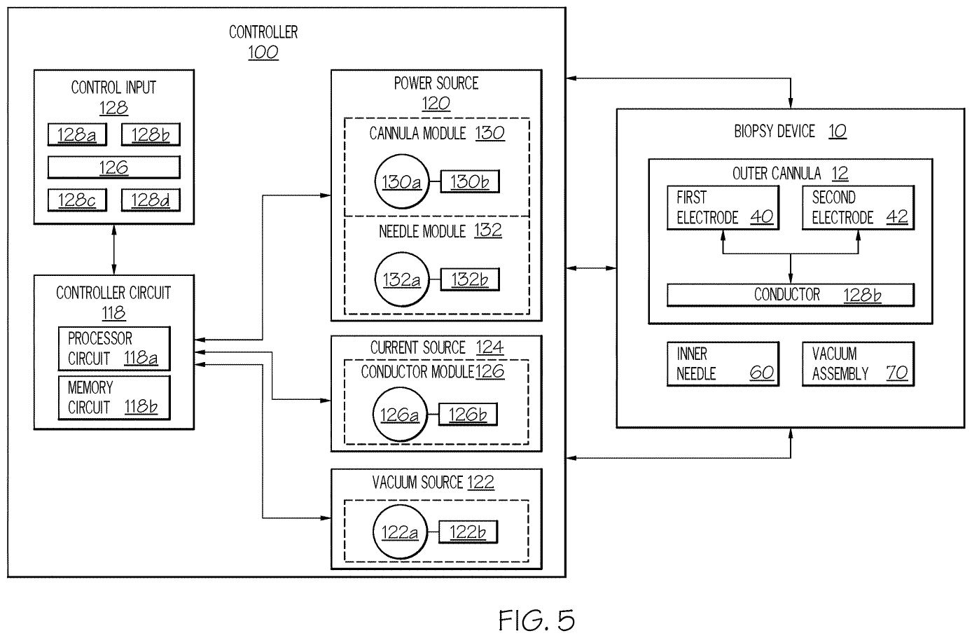

Referring now to FIG. 5, a schematic view of a controller 100 for operating the biopsy device 10 is illustrated. In some embodiments, the controller 100 may be configured to translate the inner needle 60 between the extended position and the retracted position, translate the outer cannula 12 between the initial position and the acquisition position, supply cutting current via the pair of conductors 50 to the first electrode 40 and the second electrode 42, and activate the vacuum assembly 70. In these embodiments, the controller 100 may include a controller circuit 118, a power source 120, a vacuum source 122, and a current source 124. The controller 100 may further include a control input 128, such as a computer, which may allow a user to operate the controller 100. In these embodiments, the control input 128 may be configured to provide automatic control of the controller 100 via a software program, or may be manually controlled via a user manipulating a user interface thereof.

Referring still to FIG. 5, the control input 128 may include a user interface (e.g., control buttons and visual/aural indicators, such as a display and/or speakers, with the control buttons providing user control over various functions of the controller 100, and with the visual/aural indicators providing visual/aural feedback of the status of one or more conditions and/or positions of components of the controller 100). The control buttons may include one or more buttons for actuating the mechanism(s) for moving the inner needle 60 between the extended position and the retracted position and/or extending the outer cannula 12 between the initial position and the acquisition position 128a, 128b and one or more buttons and/or knobs 128c, 128d for adjusting the frequency and amplitude of the electrical current supplied by an voltage or current source to the electrical pathway formed between the first electrode 40 and the second electrode 42.

Controller circuit 118 is electrically and communicatively coupled to power source 120, vacuum source 122, current or voltage source 124, and control input 128, such as by one or more wires or circuit traces. Controller circuit 118 may be assembled on an electrical circuit and may include, for example, a processor circuit 118a and a memory circuit 118b.

Processor circuit 118a has one or more programmable microprocessors and associated circuitry, such as an input/output interface, buffers, memory, etc. Memory circuit 118b is communicatively coupled to processor circuit 118a, e.g., via a bus circuit, and is a non-transitory electronic memory that may include volatile memory circuits, such as random access memory (RAM), and non-volatile memory circuits, such as read only memory (ROM), electronically erasable programmable ROM (EEPROM), flash memory, etc. Controller circuit 118 may be formed as one or more Application Specific Integrated Circuits (ASIC).

Controller circuit 118 is configured via software and/or firmware residing in memory circuit 118b to execute program instructions to perform functions associated with the biopsy device 10. For example, the controller circuit 118 may translate the outer cannula 12 between the initial position and the acquisition position, translate the inner needle 60 between the extended position and the retracted position, activate and adjust the vacuum assembly 70, and/or activate and adjust the cutting current provided by the current or voltage source to the pair of conductors 50.

Referring still to FIG. 5, the power source 120 may include, for example, a cannula module 130 and a needle module 132. In these embodiments, each of the cannula module 130 and the needle module 132 may be electrically and controllably coupled to the controller circuit 118. As provided herein, the cannula module 130 and the needle module 132 may be electrically coupled to the controller circuit 118 by way of electrical wiring or any other suitable electrical connections, such that the user inputs on the control input 128 may be relayed to the controller circuit 118 and used to control the power delivered by the power source 120 to the cannula module 130 and the needle module 132.

In these embodiments, the cannula module 130 may include a power source 130a to which an electric lead 130b is attached. For example, in embodiments in which the outer cannula 12 is translated between the initial position and the acquisition position using an electromechanical mechanism, the power source 130a may drive the outer cannula 12 such that the outer cannula 12 rotates and advances from the initial position to the acquisition position. Similarly, the needle module 132 may include a power source 132a to which an electric lead 132b is attached. For example, in embodiments in which the inner needle 60 is translated using an electromechanical mechanism, the power source 130a may cause the inner needle 60 to translate from the extended position to the retracted position, or vice versa.

Referring still to FIG. 5, current or voltage source 124 may include a conductor module 126. The conductor module 126 may include, for example, a power supply 126a that supplies the cutting current to the first electrode 40 and/or second electrode 42 via an electric lead 126b. In these embodiments, the current or voltage source 124 may vary the cutting current by adjusting the power provided to the first and second electrodes 40, 42 from the power supply 126a.

As noted herein, the controller 100 may further include a vacuum source 122. In these embodiments, the vacuum source 122 may include a power supply 122a, that provides power to the vacuum assembly via an electric lead 122b. When power is supplied from the power supply 122a to the vacuum assembly 70, the vacuum assembly may generate negative pressure within the outer cannula 12 of the biopsy device 10, such that tissue acquired during a biopsy procedure is drawn into the sample basket of the vacuum assembly 70.

Referring still to FIG. 5, in the embodiments described herein, the controller 100 may be electrically and operably connected to the outer cannula 12 and the inner needle 60 via the proximal end 24 of the body 20 of the outer cannula 12. For example, once the biopsy device 10 is positioned at a target site and the inner needle 60 has created an incision at the target site, the needle module 132 may be operated to cause the inner needle to retract from the extended position to the retracted position. Once the inner needle 60 is moved to the retracted position, the cannula module 130 may be operated to cause the outer cannula 12 to rotate such that the outer cannula 12 advances from the initial position to the acquisition position. As the outer cannula 12 advances to the acquisition position, the sharpened edge of the distal end of the outer cannula may burrow into the target site, creating a tissue core that is enclosed within the passageway of the outer cannula, as has been described in detail herein. Once the outer cannula 12 is advanced to the acquisition position, the current or voltage source 124 may activate the conductor module 126, such that the power supply 126a provides power to the first electrode 40 and the second electrode 42, and current passes between the first electrode 40 and the second electrode 42. As current passes between the first electrode 40 and the second electrode 42, an electrical pathway is formed between the first electrode 40 and the second electrode 42 and the tissue sample. As has been described herein, the current generated by the conductor module 126 may be adjusted such that the frequency of the electrical pathway generated between the first and second electrodes 40, 42 is sufficient to sever the tissue core from the target site. With the tissue core detached from the target site, the current source 124 may be deactivated, and the vacuum source 122 may be activated, such that the vacuum assembly 70 generates negative pressure within the outer cannula 12 to draw the tissue core through the outer cannula and into the sample basket of the vacuum assembly, at which point the biopsy device 10 may be safely removed from the target site.

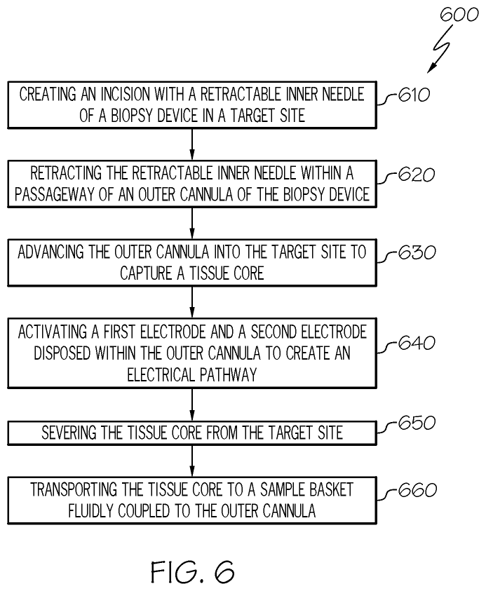

Turning now to FIG. 6, an illustrative method 600 of performing a biopsy is disclosed. In embodiments, the method 600 includes creating an incision at a target site with a retractable needle of a biopsy device, as depicted at block 610. In these embodiments, the biopsy device may further include an outer cannula a body having a proximal end, a sharpened distal end, and at least one wall defining a passageway disposed about the retractable inner needle.

Once the incision has been created at the target site, the method moves to block 620, which involves retracting the inner needle within the passageway of the outer cannula, such that the sharpened distal end of the outer cannula is exposed. As has been noted herein, in these embodiments, the sharpened distal end of the outer cannula may include a beveled, serrated, or otherwise sharpened end that is capable of perforating tissue at the target site.

With the sharpened distal end of the outer cannula exposed, the method moves to block 630, which involves advancing the sharpened distal end of the outer cannula into the target site. In these embodiments, the outer cannula may be advanced into the target site in a longitudinal direction, such that the sharpened distal end cuts a tissue core from the target site. As previously discussed herein, advancement of the outer cannula may be achieved by rotating the outer cannula in the longitudinal direction. As the sharpened distal end cuts the tissue core, the tissue core may be received by the passageway of the outer cannula, as has been described in detail herein.

Referring still to FIG. 6, the method further involves activating a first electrode and a second electrode embedded within an outer surface of the sharpened distal end of the outer cannula, as depicted at block 640. In these embodiments, the first electrode and the second electrode may be activated by supplying a voltage to the first electrode and the second electrode, such that a cutting current flows between the first electrode, the second electrode, and a conductor embedded within the inner surface of the outer cannula, thereby creating an electrical pathway between the components.

With the electrical pathway formed between the first electrode and the second electrode, the method moves to block 650, which involves severing the tissue core from the target site using the electrical pathway. In these embodiments, it should be understood that the method steps depicted at block 640 and block 650 may be performed simultaneously, such that the tissue core is severed from the target site upon formation of the electrical pathway between the first electrode and the second electrode.

Once the tissue core is severed from the target site, in some embodiments, the method may optionally involve transporting the tissue core through the passageway of the outer cannula and into a sample basket fluidly coupled to the proximal end of the outer cannula, as depicted at block 660.

As should be appreciated in view of the foregoing, a biopsy device is described herein. The biopsy device includes an outer cannula having a sharpened distal edge, a first electrode, a second electrode, and a conductor, and an inner needle is disposed within the outer cannula such that the outer cannula can rotate and extend in a longitudinal direction. By rotating the outer cannula between an initial position and an acquisition position it is possible to acquire larger tissue cores via a single biopsy incision than those currently achieved by traditional biopsy devices. Furthermore, the rotation (e.g., manual or mechanical) of the sharpened distal edge of the outer cannula allows for the biopsy device to be utilized in target sites that include calcified and/or particularly dense tissue that are prone to binding traditional biopsy devices.

Embodiments may be further described with references to the following numbered clauses:

Clause 1. A biopsy device comprising: an outer cannula comprising: a body having a distal end, a proximal end, and at least one wall defining a passageway through the body, the distal end being open and having a sharpened edge; and a first electrode and a second electrode proximal the distal end of the body; and an inner needle configured to be housed within the passageway defined by the outer cannula, the inner needle comprising a piercing tip that extends out of the distal end of the outer cannula in an extended configuration, the inner needle being retractable from the extended configuration into the passageway at a target site to expose the sharpened edge of the outer cannula to the target site such that the sharpened edge is configured to cut a tissue core and the passageway is configured to receive the tissue core drawn into the passageway via the distal end, wherein an electrical pathway is configured to be formed between the first electrode and the second electrode when a voltage is applied between the first and second electrode to sever the tissue core from the target site.

Clause 2. The biopsy device of clause 1, further comprising a first conductor and a second conductor positioned on the outer cannula.

Clause 3. The biopsy device of any of clauses 1 or 2, wherein the first electrode and the second electrode are diametrically opposed.

Clause 4. The biopsy device of any of clauses 1-3, further comprising a voltage or current source connectable to the first and second electrodes, the voltage or current source configured to supply a cutting current between 400 mA-600 mA.

Clause 5. The biopsy device of any of clauses 1-4, wherein the first electrode and the second electrode are arranged in a bipolar configuration.

Clause 6. The biopsy device of any of clauses 1-5, wherein the at least one wall of the outer cannula further comprises a slot, and the inner needle is retracted within the slot when the inner needle is in the retracted position.

Clause 7. The biopsy device of any of clauses 1-6, further comprising an inner cannula disposed within the outer cannula, wherein the outer cannula is rotatable about the inner cannula when the inner needle is in the retracted position.

Clause 8. The biopsy device of any of clauses 1-7, further comprising a vacuum assembly having a sample basket, wherein the vacuum assembly is coupled to the proximal end of the outer cannula and configured to apply a negative pressure to the passageway to transport the tissue core through the passageway of the outer cannula and into the sample basket.

Clause 9. The biopsy device of any of clauses 1-8, wherein the cutting current has a frequency between than 300 kHz and 4 MHz.

Clause 10. The biopsy device of any of clauses 1-9, wherein the sharpened edge of the outer cannula is serrated or beveled.

Clause 11. A biopsy device comprising: an outer cannula comprising: a body having a distal end, a proximal end, and at least one wall defining a passageway through the body, the distal end being open and having a sharpened edge; and a first electrode and a second electrode proximal the distal end of the body; an inner needle housed within the passageway defined by the outer cannula, the inner needle comprising a piercing tip that extends out of the distal end of the outer cannula in an extended configuration, the inner needle being retractable from the extended configuration in the passageway at a target site to expose the sharpened edge of the outer cannula to the target site such that the sharpened edge is configured to cut a tissue core and the passageway is configured to receive the tissue core drawn into the passageway via the distal end; and a vacuum assembly having a sample basket fluidly coupled to the proximal end of the outer cannula; wherein an electrical pathway is configured to be formed between the first electrode and the second electrode when a voltage is applied between the first electrode and the second electrode to sever the tissue core at the target site.

Clause 12. The biopsy device of clause 11, further comprising a first conductor and a second conductor positioned on the outer cannula.

Clause 13. The biopsy device of clauses 11 or 12, wherein the first electrode and the second electrode are diametrically opposed.

Clause 14. The biopsy device of any of clauses 11-13, further comprising a voltage or current source connectable to the first electrode and the second electrode, the voltage or current source configured to supply a cutting current between 400 mA-600 mA.

Clause 15. The biopsy device of any of clauses 11-14, wherein the inner needle is retractable within the passageway between an extended position and a retracted position, such that the inner needle extends beyond the distal end of the outer cannula in the extended position.

Clause 16. The biopsy device of any of clauses 11-15, wherein the sharpened edge of the outer cannula is serrated.

Clause 17. A method of performing a biopsy comprising: creating an incision with a retractable inner needle of a biopsy device in a target site, the biopsy device further comprising an outer cannula including a body having a proximal end, a sharpened distal end, and at least one wall defining a passageway disposed about the retractable inner needle; retracting the retractable inner needle within the passageway of the outer cannula such that the sharpened distal end of the outer cannula is exposed; advancing the sharpened distal end of the outer cannula in a longitudinal direction into the target site such that the sharpened distal end cuts a tissue core and the passageway of the outer cannula receives the tissue core; activating a first electrode and a second electrode embedded within an outer surface of the sharpened distal end of the outer cannula such that an electrical pathway is created between the first electrode and the second electrode; and severing the tissue core at the target site using the electrical pathway between the first electrode and the second electrode.

Clause 18. The method of clause 17, further comprising transporting the tissue core severed at the target site through the passageway of the outer cannula and to a sample basket fluidly coupled to the proximal end of the outer cannula.

Clause 19. The method of any of clauses 17-18, further comprising rotating the outer cannula in the longitudinal direction while the sharpened distal end of the outer cannula is advancing in the longitudinal direction.

Clause 20. The method of any of clauses 17-19, wherein severing the tissue core at the target site further comprises passing a cutting current between the first electrode and the second electrode using a conductor embedded on the inner surface of the outer cannula.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A biopsy device comprising:
an outer cannula comprising:
a body having a distal end, a proximal end, and at least one wall defining a passageway through the body, the distal end being open and having a sharpened edge; and
a first electrode and a second electrode proximal the distal end of the body; and
an inner needle configured to be housed within the passageway defined by the outer cannula, the inner needle comprising a piercing tip that extends out of the distal end of the outer cannula in an extended configuration, the inner needle being retractable from the extended configuration into the passageway at a target site to expose the sharpened edge of the outer cannula to the target site such that the sharpened edge is configured to cut a tissue core and the passageway is configured to receive the tissue core drawn into the passageway via the distal end,
wherein an electrical pathway is configured to be formed between the first electrode and the second electrode when a voltage is applied between the first and second electrode to sever the tissue core from the target site.

2. The biopsy device of claim 1, further comprising a first conductor and a second conductor positioned on the outer cannula.

3. The biopsy device of claim 1, wherein the first electrode and the second electrode are diametrically opposed.

4. The biopsy device of claim 1, further comprising a voltage or current source connectable to the first and second electrodes, the voltage or current source configured to supply a cutting current between 400 mA-600 mA.

5. The biopsy device of claim 1, wherein the first electrode and the second electrode are arranged in a bipolar configuration.

6. The biopsy device of claim 1, wherein the at least one wall of the outer cannula further comprises a slot, and the inner needle is retracted within the slot when the inner needle is in the retracted position.

7. The biopsy device of claim 1, further comprising an inner cannula disposed within the outer cannula, wherein the outer cannula is rotatable about the inner cannula when the inner needle is in the retracted position.

8. The biopsy device of claim 1, further comprising a vacuum assembly having a sample basket, wherein the vacuum assembly is coupled to the proximal end of the outer cannula and configured to apply a negative pressure to the passageway to transport the tissue core through the passageway of the outer cannula and into the sample basket.

9. The biopsy device of claim 1, wherein the cutting current has a frequency between 300 kHz and 4 MHz.

10. The biopsy device of claim 1, wherein the sharpened edge of the outer cannula is serrated or beveled.

11. A biopsy device comprising:
an outer cannula comprising:
a body having a distal end, a proximal end, and at least one wall defining a passageway through the body, the distal end being open and having a sharpened edge; and
a first electrode and a second electrode proximal the distal end of the body;
an inner needle housed within the passageway defined by the outer cannula, the inner needle comprising a piercing tip that extends out of the distal end of the outer cannula in an extended configuration, the inner needle being retractable from the extended configuration in the passageway at a target site to expose the sharpened edge of the outer cannula to the target site such that the sharpened edge is configured to cut a tissue core and the passageway is configured to receive the tissue core drawn into the passageway via the distal end; and
a vacuum assembly having a sample basket fluidly coupled to the proximal end of the outer cannula;
wherein an electrical pathway is configured to be formed between the first electrode and the second electrode when a voltage is applied between the first electrode and the second electrode to sever the tissue core at the target site.

12. The biopsy device of claim 11, further comprising a first conductor and a second conductor positioned on the outer cannula.

13. The biopsy device of claim 11, wherein the first electrode and the second electrode are diametrically opposed.

14. The biopsy device of claim 11, further comprising a voltage or current source connectable to the first electrode and the second electrode, the voltage or current source configured to supply a cutting current between 400 mA-600 mA.

15. The biopsy device of claim 11, wherein the inner needle is retractable within the passageway between an extended position and a retracted position, such that the inner needle extends beyond the distal end of the outer cannula in the extended position.

16. The biopsy device of claim 11, wherein the sharpened edge of the outer cannula is serrated.

17. A method of performing a biopsy comprising:
creating an incision with a retractable inner needle of a biopsy device in a target site, the biopsy device further comprising an outer cannula including a body having a proximal end, a sharpened distal end, and at least one wall defining a passageway disposed about the retractable inner needle;
retracting the retractable inner needle within the passageway of the outer cannula such that the sharpened distal end of the outer cannula is exposed;
advancing the sharpened distal end of the outer cannula in a longitudinal direction into the target site such that the sharpened distal end cuts a tissue core and the passageway of the outer cannula receives the tissue core;
activating a first electrode and a second electrode embedded within an outer surface of the sharpened distal end of the outer cannula such that an electrical pathway is created between the first electrode and the second electrode; and
severing the tissue core at the target site using the electrical pathway between the first electrode and the second electrode.

18. The method of claim 17, further comprising transporting the tissue core severed at the target site through the passageway of the outer cannula and to a sample basket fluidly coupled to the proximal end of the outer cannula.

19. The method of claim 17, further comprising rotating the outer cannula in the longitudinal direction while the sharpened distal end of the outer cannula is advancing in the longitudinal direction.

20. The method of claim 17, wherein severing the tissue core at the target site further comprises passing a cutting current between the first electrode and the second electrode using a conductor embedded on the inner surface of the outer cannula.
